(19) United States
(12) Patent Application Publication
(10) Pub. No.: US 2010/0196469 A1
(43) Pub. Date: Aug. 5, 2010

(54) Pharmaceutical Formulation containing an HMG-COA Reductase Inhibitor and method for the preparation thereof

(75) Inventors: Evangelos Karavas, Pallini Attikis (GR); Efthimios Koutris, Pallini Attikis (GR); Elli Ioannidou, Pallini Attikis (GR); Vicky Samara, Pallini Attikis (GR); Dimitrios Bikiaris, Thessaloniki (GR)

Correspondence Address: AKC PATENTS, 215 GROVE ST., NEWTON, MA, 02466, US

(73) Assignee: PHARMATHEN S.A., PALLINI-ATTIKIS (GR)

(21) Appl. No.: 12/663,513

(22) PCT Filed: Jun. 25, 2007
(86) PCT No.: PCT/EP2007/005568

§ 371 Date: Dec. 7, 2009

(51) Int. Cl. A61K 9/48; A61K 31/40; A61K 31/405; A61P 9/10; A61K 9/52

(52) U.S. Cl. 424/457; 514/423; 514/419; 424/451

(57) ABSTRACT

The present invention relates to the formulation of solid dosage forms comprising a therapeutically effective amount of an HMG-CoA reductase inhibitor, and especially Atorvastatin or Fluvastatin or salts thereof, in combination with colloidal clay such as Attapulgite, and a process for the preparation thereof by direct compression.

### TECHNICAL FIELD OF THE INVENTION

The present invention relates to improved pharmaceutical formulations for oral administration comprising a therapeutically effective quantity of an HMG-CoA reductase inhibitor, and more particularly Atorvastatin, Fluvastatin or pharmaceutical acceptable salts thereof in combination with an effective amount of colloidal clay such as Attapulgite, as a stabilizer and a method for the preparation thereof.

### BACKGROUND OF THE INVENTION

In order to achieve an effective treatment and avoid side effects to the patient, a pharmaceutical dosage form should be stable and contain low levels of impurities for a long period of time. Impurities can not be totally eliminated during production of the active substance but generally are in very low levels. Degradation products of the active ingredient may also occur during the preparation of the final pharmaceutical formulation, by interaction with other ingredients or by various environmental factors such as temperature, moisture, pH and light. So the process of preparation of a pharmaceutical composition and the pharmaceutical excipients should be chosen very carefully.

HMG-CoA reductase inhibitors, commonly known as “statins”, act through the inhibition of 3-hydroxy-3-methylglutaryl-coenzyme A (HMG-CoA) reductase. This enzyme catalyzes the conversion of HMG-CoA to mevalonate, an early and rate-limiting step in cholesterol biosynthesis. Statins are useful in the treatment of hypercholesterolemia and associated diseases but are extremely susceptible to degradation at pH below 8. Statins at pH below 8 and particularly in acidic conditions, undergo elimination or isomerization or oxidation or re-crystallization reactions to form conjugated unsaturated aromatic compounds, as well as the threo isomer, the corresponding lactones and other degradation products. Statins are particularly sensitive to an acidic environment (a low pH environment), in which hydroxyl acids are degraded into lactone. The tendency of HMG-CoA reductase inhibitors to degrade may be accelerated by possible interactions with other active ingredients or excipients present in the composition.

Fluvastatin sodium, is the [R*,S*-(E)]-(±)-7-[3-(4-fluorophenyl)-1-(1-methylethyl)-1H-indol-2-yl]-3,5-dihydroxy-6-heptenoic acid, monosodium salt and Atorvastatin calcium, is the [R—(R*,R*)]-2-(4-fluorophenyl)-b,d-dihydroxy-5-(1-methylethyl)-3-phenyl-4[(phenylamino)carbonyl]-1H -pyrrole-1-heptanoic acid, calcium salt (2:1) trihydrate. Fluvastatin and Atorvastatin are two statins particularly useful in therapeutics but prone to degradation reactions. The degradation of the active ingredient results in reduced effectiveness and treatment failure.

Furthermore, the stability of pharmaceutical compositions containing a HMG-CoA reductase inhibitor and in particular, Atorvastatin or Fluvastatin or salts thereof can also be influenced by the selection of the excipients. Moreover, the bioavailability and the release rate of the pharmaceutical dosage can also be enhanced by the selection of the excipients.

Various methods are already known for the industrial preparation of oral dosage forms comprising a HMG-CoA reductase inhibitor e.g. Atorvastatin or Fluvastatin or salts thereof, as an active ingredient due to its useful therapeutical properties. However, the prior art has encountered substantial difficulties in the production of the oral solid formulations of a desirable stability due to the degradation of said active ingredient.

EP 0 547 000 discloses a stabilized pharmaceutical composition which comprises a statin and an alkaline stabilizing medium capable of imparting a pH of at least 8 to an aqueous solution or dispersion of the composition.

EP 1 148 872 discloses a stable solid pharmaceutical formulation comprising a statin and a buffering agent, such as a carbonate buffer or phosphate buffer, capable of adjusting the pH of the total formulation in the range from 7 to 11.

Furthermore, in EP 1 292 293 is disclosed a composition comprising a homogenous mixture of a statin with a buffering or basifying substance obtained by co-crystallization and/or co-precipitation of the statin and the buffering or basifying substance.

Although each of the above patents represents an attempt to overcome the instability problems associated with pharmaceuticals compositions for immediate or sustained release comprising a HMG-CoA reductase inhibitor, there still exists a need for improving the stability and release rate of such pharmaceutical compositions without producing unwanted pharmaceutical effects and with low production costs.

### SUMMARY OF THE INVENTION

It is, therefore, an object of the present invention to provide an improved stable solid dosage formulation for oral administration containing a HMG-CoA reductase inhibitor, and in particular Atorvastatin or Fluvastatin or pharmaceutical acceptable salts thereof as an active ingredient, which overcomes the deficiencies of the prior art and avoids the degradation of the active substance.

It is another object of the present invention to provide a solid pharmaceutical dosage formulation for oral administration containing a HMG-CoA reductase inhibitor, and in particular Atorvastatin or Fluvastatin or pharmaceutical acceptable salts thereof as an active ingredient, having an increased chemical stability of the active ingredient, sufficient self-life and good pharmacotechnical properties.

Moreover, it is another object of the present invention to provide a solid dosage formulation for oral administration containing a HMG-CoA reductase inhibitor, and in particular Atorvastatin or

Fluvastatin or salts thereof as an active ingredient, which can be prepared in dosage forms of different strength by proportionally adjusting the quantities of the excipients and the active ingredient, thereby providing a pharmacotechnical linearity, without affecting the dissolution profile and bioavailability of the active ingredient.

A further aspect of the present invention is to provide a method for the preparation of a stable solid dosage formulation for oral administration containing a HMG-CoA reductase inhibitor, and in particular Atorvastatin or Fluvastatin or pharmaceutical acceptable salts thereof as an active ingredient, thereby stabilizing said active ingredient and improving the flow properties and the pharmacotechnical characteristics of the formulation.

In accordance with the above objects of the present invention, a pharmaceutical composition for oral administration is provided comprising an HMG-CoA inhibitor or a pharmaceutical acceptable salt thereof as an active ingredient, and an effective amount of colloidal clay, such as Attapulgite as a stabilizer, to inhibit hydrolysis and/or isomerization and/or elimination and/or oxidation and/or re-crystallization.

According to another embodiment of the present invention, a process for the preparation of solid dosage form for oral administration such as tablets, capsules and sachets, containing a HMG-CoA reductase inhibitor, and in particular Atorvastatin or Fluvastatin or pharmaceutical acceptable salts thereof as an active ingredient and an effective amount of colloidal clay such as Attapulgite as a stabilizer to inhibit hydrolysis and/or isomerization and/or elimination and/or oxidation and/or re-crystallization is provided, which comprises:
Forming a homogenous mixture by mixing the total quantity of said active ingredient with a portion of the total quantity of said colloidal clay such as Attapulgite, and at least one optionally excipient such as a binder, a diluent, a disintegrant and/or a glidant and mixing until uniform;
Sieving the above mixture through a sieve;
Adding to the sieved mixture the total quantities of at least one optionally excipient such as a binder, a diluent, a disintegrant and/or a glidant and mixing until uniform;
Admixing the remaining portion of the total quantity of said colloidal clay such as Attapulgite until uniform;
Subsequently adding a lubricant and forming a homogenous mixture, and
Formulating the resulting mixture in a solid dosage form either by compressing it into a desired tablet form or by filling capsules or sachets.

Alternative processes for the preparation of the pharmaceutical composition according to the present invention are also defined in independent claim 15.

Further preferred embodiments of the present invention are defined in dependent claims 2 to 13 and 16 to 20.

Other objects and advantages of the present invention will become apparent to those skilled in the art in view of the following detailed description.

### BRIEF DESCRIPTION OF THE DRAWINGS

FIG. 1 shows X-RD spectrum of Atorvastatin active ingredient used for the preparation of the composition according to the present invention.

FIG. 2 shows X-RD spectrum of placebo tablets prepared according to the present invention.

FIG. 3 shows X-RD spectrum of Atorvastatin 80 mg tablets directly after preparation according to the present invention.

FIG. 4 shows a comparative X-RD spectrum of Atorvastatin active ingredient, placebo and Atorvastatin 80 mg tablets prepared according to the present invention

### DETAILED DESCRIPTION OF THE INVENTION

For the purposes of the present invention, a pharmaceutical composition comprising an active ingredient (HMG-CoA reductase inhibitor e.g. Atorvastatin or Fluvastatin or salts thereof) is considered to be “stable” if said ingredient degradates less or more slowly than it does on its own and/or in known pharmaceutical compositions.

An excipient is considered to be “incompatible” with an active ingredient (HMG-CoA reductase inhibitor e.g. Fluvastatin or Atorvastatin or salts thereof) if it promotes the degradation of said active ingredient, that is to say, if said active ingredient (HMG-CoA reductase inhibitor e.g. Fluvastatin or Atorvastatin or salts thereof) degrades more or faster in the presence of said excipient when compared with the degradation of said active ingredient (HMG-CoA reductase inhibitor e.g. Fluvastatin or Atorvastatin or salts thereof) on its own. The terms “incompatibility”, “compatible” and “compatibility” are defined accordingly.

The active ingredient (HMG-CoA reductase inhibitor e.g. Fluvastatin or Atorvastatin or salts thereof) contained in a dosage form is “bioavailable”, if when administered in a dosage form is released from the dosage form, absorbed and reaches, at least the same, concentration levels in plasma as any of the marketed products containing the same quantity of the same active ingredient and intended for the same use.

Although the pharmaceutical composition may be in various forms, the preferred solid forms are tablets, capsules and caplets.

As already mentioned certain HMG-CoA reductase inhibitors are susceptible to degradation/oxidation/hydrolysis and their tendency gets stronger when they are formulated and mixed with excipients or other active substances.

One of the main disadvantages of the HMG-CoA reductase inhibitors is the fact that, they are very labile to acidic pH environment, and consequently many limitations concerning the choice of excipients are raised.

Moreover, the manufacturing process should also be very carefully determined because relatively high concentrations of lubricant and/or glidant reduce crashing strength and increase disintegration time especially when associated with prolonged mixing times.

Furthermore, it is already known either to include alternative excipients as lubricants with or without stabilizing agents, or to use more complicated formulations and/or manufacturing processes.

It has been surprisingly found that the object of the present invention is achieved by employing colloidal clay such as Attapulgite as a stabilizer. In fact, when Attapulgite is incorporated in a pharmaceutical composition according to the present invention, it is not necessary to employ an additional buffering or alkaline agent in order to avoid the degradation of statins.

Attapulgite is a purified native hydrated magnesium aluminum silicate consisting of the clay mineral polygorskite. Attapulgite is widely used as an adsorbent in solid dosage forms. Colloidal clays such as Attapulgite absorb considerable amounts of water to form gels and in concentrations of 2-5% w/v usually form oil-in-water emulsions. Activated Attapulgite, which is Attapulgite that has been carefully heated to increase its absorptive capacity, is used therapeutically as an adjunct in the management of diarrhoea.

When Attapulgite is incorporated in a pharmaceutical composition according to the present invention, adsorb considerable amounts of water, swells and preserves this amount, providing excellent storage stability, caused by the reduction of the free water mobility, derived from the excipients or the environment, into the composition. Said system protects the active ingredient from hydrolysis and/or oxidation and/or elimination and/or isomerization. Thus, Attapulgite serves as a protective barrier, isolating the active ingredient against humidity and/or air oxygen and/or a low pH environment. The protection of the active ingredient may be attributed to the high hygroscopic character of Attapulgite, related to its structure. This, however, unexpectedly does not affect the dissolution rate of the active substance as it is used in such a proportion that its hygroscopic properties prevail and therefore excellent bioavailability is achieved.

In that respect, the stability of Atorvastatin can be increased by far in solid state even in the presence of moisture. In addition to that, by releasing it in aqueous medium, the concentration of Atorvastatin calcium as a free salt will be reduced, thus giving less time for the lactonization process to occur.

One of the main objects of the present invention was to prepare a product with an acceptable stability. For this reason samples of Atorvastatin alone and Atorvastatin with Attapulgite were exposed to compatibility tests at a temperature of 40° C. and relative humidity of 75% for a certain period of time.

The results show an acceptable compatibility between Atorvastatin and Attapulgite.

The specific tests and results are described in the TABLE 1.

TABLE 1
STABILITY AT 40° C. TEMPERATURE
AND 75% RELATIVE HUMIDITY

| | 40° C./75% RH | |
|---|---|---|
| | | Atorvastatin- |
| | Atorvastatin | Attapulgite |
| Atorvastatin Amide NMT 0.3% | 0.05% | 0.05% |
| Desfluoro NMT 0.3% | 0.07% | 0.07% |
| Diastereoisomer NMT 0.3% | ND | ND |
| O-Methyl NMT 0.3% | ND | ND |
| Lactone NMT 2% | 0.34% | 0.33% |
| Methyl Ester NMT 0.3% | 0.01% | 0.02% |
| Atorvastatin Diepoxide NMT 0.6% | 0.20% | 0.17% |
| Diketo1,2-Diol + Diketoepoxide 1.20% | 0.39% | 0.43% |
| Total | 1.29% | 1.32% |

It has been confirmed through several tests such as compatibilities studies, X-RD analysis, that the active ingredient and the formulation of the present invention remain stable.

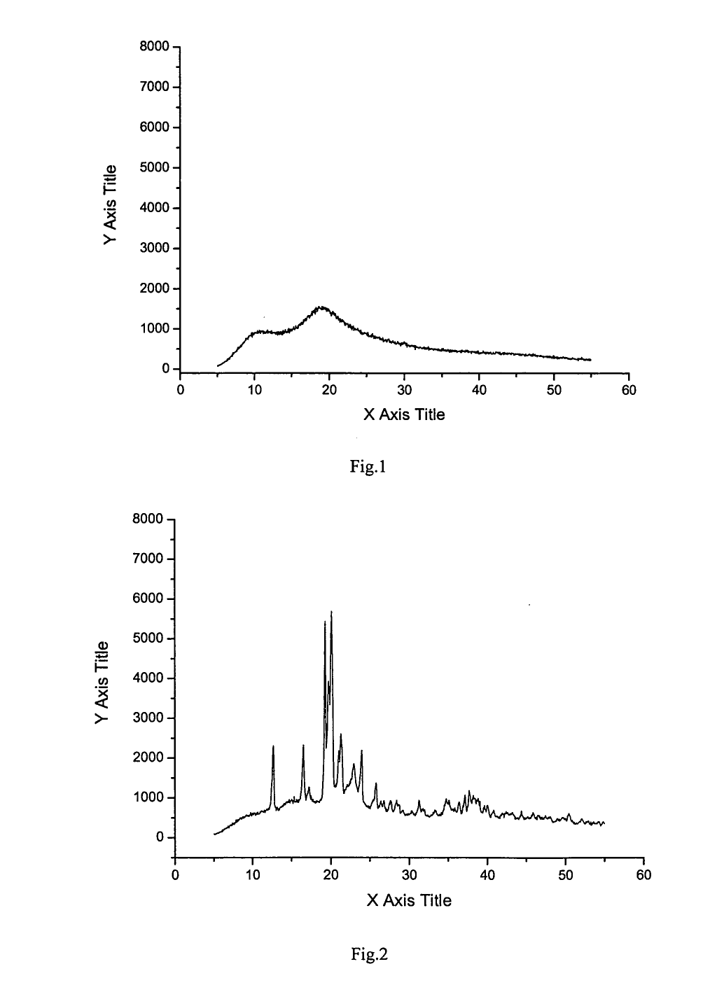

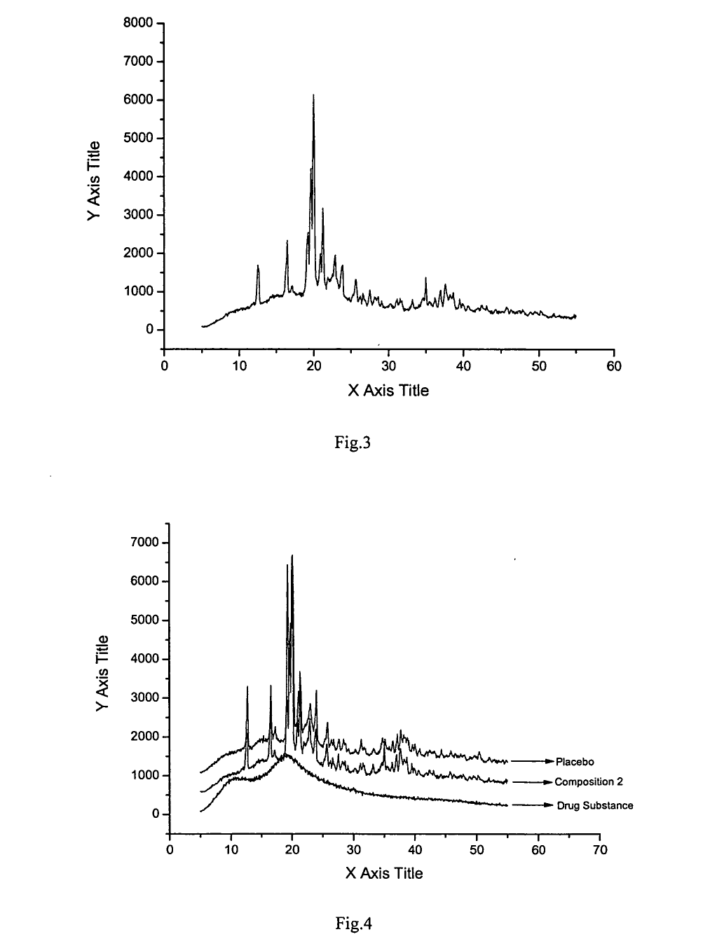

In addition, as shown in FIGS. 1, 3 and 4 by the X-RD analysis Atorvastatin active ingredient is completely amorphous since only a broad band is recorded with a maximum at around 2θ=19 deg. The mixture of an amorphous statin and Attapulgite does not promote the crystallization of said Statin (Atorvastatin remains amorphous), as it has been confirmed by X-RD analysis wherein all the recorded characteristic broad band were unchanged after 6 months storage in accelerated conditions (40° C. and 75% RH). The main peaks obtained for Atorvastatin tablets prepared according to the present invention, are at approximately 2θ=12.6, 16.5, 19.0, 19.5, 20.0, 21.0, 21.3, 22.9, 23.9, 25.6, 35.0 degrees which are also found in the placebo tablets (FIG. 2). The crystal properties of amorphous active ingredient remain also unchanged after six months in the same conditions when the mixture is incorporated in a pharmaceutical composition with other excipients. No peaks corresponding to any crystalline form of Atorvastatin are observed before or after storage indicating that the mixture is stabilized.

Moreover, any excipient may optionally be added to the above composition, provided that they are compatible with the active ingredient of the composition, in order to overcome problems associated with the poor flow properties and unfavorable pharmacotechnical characteristics of these substances, and in order to increase the stability of the drug and the self-life of the pharmaceutical product, and provide a product exhibiting excellent bioavailability.

The present invention can be applied in the formulation of tablets, orally disintegrating tablets, capsules, caplets, sachets or other solid dosage forms for oral administration of an active ingredient having stability problems.

Another essential advantage of the present invention is that the solid dosage form according to the present invention ensures excellent bioavailability of the active ingredient. Furthermore, it is possible to prepare dosage forms of different strength using appropriate quantity of the same composition, thereby limiting the cost of production and minimizing the number, and consequently the cost, of clinical studies required for the approval of the product by the authorities.

The manufacturing process for preparation according to the present invention is simpler and inexpensive in comparison to any other conventional method.

Therefore, in a first embodiment, the present invention provides a pharmaceutical composition comprising from about 0.5% to 30% by weight of Atorvastatin or the salt thereof and from about 1% to 12% by weight of Attapulgite. The weight ratio of the Atorvastatin to Attapulgite is preferably 30: 1 to 1:24.

Preferred pharmaceutical compositions according to the present invention comprise approximately 0.5% to 20%, more preferably 1% to 15% and most preferably 3% to 10% by weight of Atorvastatin or the salt thereof.

More preferred pharmaceutical compositions according to the present invention comprise approximately 1% to 10%, more preferably 2% to 9% and most preferably 4% to 7% by weight of Attapulgite.

The preferred pharmaceutical compositions are in the form of solid dosage forms such as tablets, orally disintegrating tablets, capsules, caplets, troches, pastilles, pills, lozenges and the like, in all shapes and sizes, coated or uncoated.

All percentages stated herein are weight percentages based on total composition weight, unless otherwise stated.

Another embodiment of the present invention is the use of the direct compression process for the preparation of solid dosage forms such as tablets containing HMG-CoA reductase inhibitor such as Atorvastatin or Fluvastatin or salts thereof, which is one of the most economical methods.

Wet granulation techniques are avoided due to increased degradation products of the active substance that may occur when an HMG-CoA reductase inhibitor is incorporated in a pharmaceutical dosage form.

The direct compression process of the present invention for the preparation of solid dosage forms for oral administration such as tablets containing HMG-CoA reductase inhibitor such as Atorvastatin or Fluvastatin or salts thereof as an active ingredient and an effective amount of colloidal clay such as Attapulgite as a stabilizer to inhibit hydrolysis and/or isomerization and/or elimination and/or oxidation and/or re-crystallization comprises:
Forming a homogenous blend by mixing the total quantity of said active ingredient with a portion of the total quantity of said colloidal clay such as Attapulgite and at least one optionally excipient such as a binder, a diluent, a disintegrant and/or a glidant an mixing until uniform;
Sieving the above mixture through a sieve;
Adding to the sieved mixture the total quantities of at least one optionally excipient such as a binder, a diluent, a disintegrant and/or a glidant and mixing until uniform;
Admixing the remaining portion of the total quantity of said colloidal clay such as Attapulgite until uniform;
Subsequently adding a lubricant and forming a homogenous mixture, and
Formulating the resulting mixture in a solid dosage form either by compressing it into a desired tablet form or by filling capsules or sachets.

The pharmaceutical compositions according to the present invention are characterized by excellent pharmacotechnical properties, such as homogeneity, flowability and compressibility.

Thanks to these properties, the solid dosage forms prepared by the above process exhibit excellent technical characteristics including disintegration time, dissolution rate, hardness, resistance to crashing, friability and stability, as better illustrated by the following measurements during the stage of the development of the products.

Namely, the pure pharmaceutical substance Atorvastatin showed a mean Carr's Index of 22.3%. However, when Attapulgite was incorporated according to the present invention, a decrease of 19% of the Carr Index was observed, which indicates an improvement of the flow properties of Atorvastatin and therefore the use of a direct compression for the final formulation.

The pharmaceutical formulations according to the present invention have excellent pharmacotechnical properties indicating the suitability of the process and of the selected excipients as well.

One of the most critical pharmacotechnical tests, is the Dissolution test as it is strongly correlated with the bioavailability of the product. For the dissolution method a Paddle Apparatus was used 75 rpm, 37° C., time 30 min, while as a dissolution medium 1000 ml of H₂O was used.

Dissolution Profiles

| MIN | Comp. 1 | Comp. 2 | Comp. 4 | Atorvastatin |
|---|---|---|---|---|
| 5 | 80.79 | 74.03 | 57.95 | 15.37 |
| 15 | 93.01 | 88.82 | 86.15 | 50.80 |
| 30 | 93.39 | 90.71 | 92.41 | 58.48 |

The most preferable compositions described below were investigated for their scalability, while a process validation was performed in order to prove the repeatability and accuracy of the manufacturing process and the proposed formulations.

The validation process showed that the compositions and the manufacturing process are suitable in order to provide a repeatable and high quality product.

One of the main objects of the present invention was to prepare a product with acceptable stability. For this reason 3 batches of each composition were exposed to normal, intermediate and accelerated stability studies according to the current ICH guidelines.

The following compositions were tested.

Compositions

| | Comp 1 | Comp 2 | Comp 3 | Comp 4 |
|---|---|---|---|---|
| | mg per | mg per | mg per | mg per |
| Ingredients | tab | tab | tab | tab |
| Internal Phase | 80 | 80 | 80 | 80 |
| Atorvastatin Ca | 82.72 | 82.72 | 82.72 | 82.72 |
| Microcelac 100 | 775.28 | 769.28 | 811.28 | 811.28 |
| Starch 1500 LM | 300.00 | 264.0 | 300.00 | 300.00 |
| Attapulgite | 36.00 | 72.0 | | |
| Mg Stearate | 6.00 | 12.00 | 6.00 | 6.00 |
| Total | 1200.0 | 1200.0 | 1200.0 | 1200.0 |

The tablets of the above compositions were exposed at a temperature of 40° C.±2° C. and relative humidity of 75%±5% without being packed in any type of container or blister. The tablets were tested in predetermined time intervals. The results for each composition are described in the stability table (TABLE 1).

TABLE 2
STABILITY AT 40° C. ± 2° C. TEMPERATURE AND
75 ± 5% RELATIVE HUMIDITY

| | Comp. | Comp. | Comp. | Comp. | |
|---|---|---|---|---|---|
| 40°/75 RH | 1 | 2 | 3 | 4 | Atorvastatin |
| Atorvastatin Amide | 0.06 | 0.04 | 0.05 | 0.07 | 0.07 |
| Desfluoro | 0.05 | 0.04 | 0.08 | 0.03 | 0.08 |
| Diastereoisomer | ND | ND | ND | ND | ND |
| O-Methyl | ND | ND | ND | ND | ND |
| Lactone | 0.56 | 0.48 | 2.10 | 0.97 | 0.45 |
| Methyl Ester | 0.07 | ND | ND | ND | ND |
| Atorvastatin | 0.26 | 0.32 | 0.32 | 0.28 | 0.34 |
| Diepoxide | | | | | |
| Diketo 1,2- | 0.73 | 0.80 | 1.62 | 0.75 | 0.69 |
| Diol + | | | | | |
| Diketoepoxide | | | | | |
| Total | 1.82 | 1.68 | 4.17 | 2.10 | 1.87 |

Subsequently, the tablets of compositions 2 were packed into white opaque, HDPE plastic containers and stored closed in appropriate stability chambers at a temperature of 75° C.+7° C. and relative humidity of 60%±5% for normal conditions and at a temperature of 30° C.±2° C. and relative humidity of 65%±5% for intermediate conditions. The results for composition 2 and of the active ingredient directly after preparation and after 3 months stability are described in the stability table below (TABLE 3).

TABLE 3
STABILITY AFTER PREPARATION AND AFTER 3 MONTHS
AT 25° C. ± 2° C. TEMPERATURE
AND 60 ± 5% RELATIVE HUMIDITY AND 30°
C. ± 2° C. TEMPERATURE AND 65 ± 5% RELATIVE HUMIDITY

| | | | |
|---|---|---|---|
| | 0 | 25° C./ | 30° C./ |
| IMPURITY | MONTHS | 60% RH | 65% RH |

ACTIVE SUBSTANCE

| | | | |
|---|---|---|---|
| Atorvastatin Amide NMT 0.3% | 0.06 | 0.06 | 0.06 |
| Desfluoro NMT 0.3% | 0.06 | 0.03 | 0.03 |
| Diastereoisomer NMT 0.3% | ND | ND | ND |
| O-Methyl NMT 0.3% | ND | ND | ND |
| Lactone NMT 2% | 0.07 | 0.05 | 0.05 |
| Methyl Ester NMT 0.3% | ND | ND | ND |
| Atorvastatin Diepoxide NMT 0.6% | 0.25 | 0.30 | 0.38 |
| Diketo1,2-Diol + | 0.84 | 0.92 | 1.10 |
| Diketoepoxide NMT 1.20% | | | |
| Unknown | 0.34 | 0.38 | 0.40 |
| Total | 1.69 | 1.96 | 2.12 |

COMPOSITION 2

| | | | |
|---|---|---|---|
| Atorvastatin Amide NMT 0.3% | 0.06 | 0.06 | 0.04 |
| Desfluoro NMT 0.3% | 0.06 | 0.04 | 0.07 |
| Diastereoisomer NMT 0.3% | ND | ND | ND |
| O-Methyl NMT 0.3% | ND | ND | ND |
| Lactone NMT 2% | 0.07 | 0.06 | 0.07 |
| Methyl Ester NMT 0.3% | ND | ND | ND |
| Atorvastatin Diepoxide NMT 0.6% | 0.30 | 0.38 | 0.43 |
| Diketo1,2-Diol + | 0.85 | 0.96 | 1.03 |
| Diketoepoxide NMT 1.20% | | | |
| Unknown | 0.34 | 0.37 | 0.36 |
| Total | 1.74 | 1.97 | 2.07 |

The results show a good stability of the product and compatibility between the drug substance and the excipients proposed by the present invention. The excellent results regarding the physicochemical characteristics, the excellent stability of the product as well as the simple and economic manufacturing process indicate the advantages of the present invention relative to the commonly used methods and excipients for the formulation of Atorvastatin and Fluvastatin.

The pharmaceutical compositions of the present invention may also contain one or more additional formulation ingredients selected from a wide variety of excipients. According to the desired properties of the composition, any number of ingredients may be selected, alone or in combination, based upon their known uses in preparation of solid dosage form compositions.

Such ingredients include, but are not limited to, diluents, binders, compression aids, disintegrants, glidants, lubricants, flavors, water scavengers, colorants, sweetener, coating agents and preservatives.

The optional excipients must be compatible with the HMG-CoA reductase inhibitor so that it does not interfere with it in the composition.

Diluents may be, for example, calcium carbonate, calcium phosphate dibasic, calcium phosphate tribasic, calcium sulfate, microcrystalline cellulose, microcrystalline silicified cellulose, powdered cellulose, dextrates, dextrose, fructose, lactitol, lactose anhydrous, lactose monohydrate, lactose dihydrate, lactose trihydrate, mannitol sorbitol, starch, pregelatinized starch, sucrose, talc, xylitol, maltose maltodextrin, maltitol.

Binders may be, for example, acacia mucilage, alginic acid, carbomer, carboxymethylcellulose calcium, carboxymethylcellulose sodium, microcrystalline cellulose, powdered cellulose, ethyl cellulose, gelatin, liquid glucose, guar gum, hydroxyethyl cellulose, hydroxypropyl cellulose, hydroxypropylmethyl cellulose, maltodextrin, methylcellulose, polydextrose, polyethylene oxide, povidone, sodium alginate, starch paste, pregelatinized starch, sucrose.

Disintegrants may be, for example, alginic acid, carbon dioxide, carboxymethylcellulose calcium, carboxymethylcellulose sodium, microcrystalline cellulose, powdered cellulose, croscarmelose sodium, crospovidone, sodium docusate, guar gum, hydroxypropyl cellulose, methylcellulose, polacrilin potassium, poloxamer, povidone, sodium alginate, sodium glycine carbonate, sodium laulyl sulfate, sodium starch glycolate, starch, pregelatinized starch.

Glidants may be, for example, calcium silicate, powdered cellulose, starch, talc, lubricants e.g. polyethylene glycol 4000, polyethylene glycol 6000, sodium lauryl sulfate, starch, talc.

Still another embodiment of the present invention is the use of Attapulgite as an agent to improve flow properties of HMG-CoA reductase inhibitor such as Atorvastatin, or Fluvastatin and/or to prevent sticking to parts of the processing machines, for example tableting machine and/or to protect and stabilize hydrolysis and/or oxidation susceptible pharmaceutical substances.

The following examples illustrate preferred embodiments in accordance with the present invention without limiting the scope or spirit of the invention:

### Examples

### Example 1

### Tablet of 80 mg Atorvastatin (Comp. 1)

80 mg tablets

| | % of total | |
|---|---|---|
| Ingredients | weight | mg per tab |
| Atorvastatin Ca | 6.89 | 82.72 |
| Microcelac 100 | 64.61 | 775.28 |
| Starch 1500 Lm | 25.00 | 300.00 |
| Attapulgite | 3.00 | 36.00 |
| Mg Stearate | 0.50 | 6.00 |
| Total | 100.00 | 1200.0 |

Tablets of the above formulation were prepared according to the following manufacturing process: Atorvastatin with the total quantity of Starch 1500 LM as a moisture absorbent and Microcellac 100 for improved flow properties, were admixed to form a homogenous mixture. The above mixture was passed through a sieve. The sieved mixture was subsequently mixed with the total quantity of Attapulgite until uniform and finally mixed with Magnesium stearate.

The final mixture was then compressed directly into tablets in a tableting machine with round punches.

The bulk mixture showed satisfactory flow and could also be filled into capsules or sachets or compressed into tablets. The later solution was selected and the produced tablets were tested for hardness, friability, disintegration, and water content. All tests were performed according to European Pharmacopoeia 5.1 and were well within the specifications. Dissolution test in 1000 ml water, 50 rpm Paddle Apparatus showed more than 85% dissolved in 30 min.

### Example 2

### Tablet of 80 mg Atorvastatin (Comp. 2)

80 mg tablets

| | % of total | |
|---|---|---|
| Ingredients | weight | mg per tab |
| Atorvastatin Ca | 6.89 | 82.72 |
| Microcelac 100 | 64.11 | 769.28 |
| Starch 1500 LM | 22.00 | 264.00 |
| Attapulgite | 6.00 | 72.00 |
| Mg Stearate | 1.00 | 12.00 |
| Total | 100.00 | 1200.0 |

Tablets of this formulation were prepared according to following manufacturing process:

a) Internal Phase: A first blend of Atorvastatin, Starch 1500 LM and ½ of the batch quantity of Attapulgite was formed and mixed until complete homogeneity. Thus a first internal moisture barrier is being formed. The above mixture was sieved and subsequently the sieved mixture was mixed with the total quantity of Microcellac 100 to complete homogeneity.

b) External Phase: The remaining portion of the batch quantity of Attapilgite was added to the first blend and mixed. Subsequently, the total quantity of lubricant Mg stearate was added and mixed for two minutes.

Thus a second moisture barrier is being formed, externally to the particles of the internal phase.

The final mixture of the composition was then compressed directly into tablets.

Tablets were produced and tested for content uniformity, disintegration, water content and dissolution proving that they are meeting the specifications.

### Example 3

### Tablet of 80 mg Atorvastatin (Comp. 3)

80 mg tablets

| | % of total | |
|---|---|---|
| Ingredients | weight | mg per tab |
| Atorvastatin Ca | 6.89 | 82.72 |
| Microcelac 100 | 67.61 | 811.28 |
| Starch 1500 Lm | 25.00 | 300.00 |
| Mg Stearate | 0.50 | 6.00 |
| Total | 100.00 | 1200.0 |

Tablets of this formulation were prepared according to the following manufacturing process: Atorvastatin, Microcellac 100 and Starch 1500 LM were admixed until complete homogeneity and subsequently wet granulated using an organic solvent. The wetted mass was then dried in hot air chamber, passed through a sieve to achieve the granule size and further mixed with Magnesium stearate. The final blend was then compressed into tablets in a tableting machine with round punches.

From the produced bulk mixture, tablets weighting 1200 mg were produced and tested for hardness, friability, disintegration, and water content and results were well within the specifications. Furthermore dissolution in 1000 ml water, 50 rpm Paddle Apparatus has been performed.

### Example 4

### Tablet of 80 mg Atorvastatin (Comp. 4)

Tablets of the formulation of example 3 were prepared according to the following manufacturing process: Atorvastatin, Microcellac 100 and Starch 1500 LM were admixed until complete homogeneity. The above mixture was sieved and then blended with Magnesium stearate. The final mixture of the composition was compressed into tablets.

While the present invention has been described with respect to the particular embodiments, it will be apparent to those skilled in the art that various changes and modifications may be made in the invention without departing from the spirit and scope thereof, as defined in the appended claims.

1. A pharmaceutical composition for oral administration comprising a HMG-CoA reductase inhibitor or a pharmaceutical acceptable salt thereof, as an active ingredient, and an effective amount of colloidal clay such as Attapulgite as a stabilizer to inhibit hydrolysis and/or isomerization and/or elimination and/or oxidation and/or re-crystallisation.

2. The pharmaceutical composition according to claim 1, wherein said colloidal clay is Attapulgite.

3. The pharmaceutical composition according to claim 2, wherein said composition does not comprise any buffering or alkaline agent.

4. The pharmaceutical composition according to claim 2, wherein the weight ratio of said HMG-CoA reductase inhibitor or salt thereof to Attapulgite is preferably from 30:1 to 1:24.

5. The pharmaceutical composition according to claim 1, wherein it comprises approximately 0.5% to 30%, preferably 0.5% to 20%, more preferably 1% to 15% and most preferably 3% to 10% by weight of said HMG-CoA reductase inhibitor or salt thereof.

6. The pharmaceutical composition according to claim 2, wherein it comprises approximately 1% to 25%, preferably 1% to 10%, more preferably 2% to 9% and most preferably 4% to 7% by weight of Attapulgite.

7. The pharmaceutical composition according to claim 6, wherein said HMG-CoA reductase inhibitor is Atorvastatin or a salt thereof.

8. The pharmaceutical composition according to claim 6, wherein said HMG-CoA reductase inhibitor is Fluvastatin or a salt thereof.

9. The pharmaceutical composition according to claim 6, wherein it further comprises at least one optionally excipient selected from the group consisting of diluents, binders, disintegrants, lubricants, and glidants.

10. The pharmaceutical composition according to claim 6, wherein said composition is in a solid dosage form such as a tablet, orally disintegrating tablet, capsule or sachet comprising an active ingredient such as Fluvastatin or Atorvastatin or salts thereof.

11. The pharmaceutical composition according to claim 6, wherein said composition comprises Atorvastatin or salt thereof Attapulgite, Starch, Microcelac and Magnesium Stearate

12. The pharmaceutical composition according to claim 6, wherein said composition is immediate release.

13. The pharmaceutical composition according to claim 6, wherein said composition is sustained release.

14. A process for the preparation of a solid dosage form for oral administration such as a tablet, capsule or sachet containing a HMG-CoA reductase inhibitor or a pharmaceutical acceptable salt thereof as an active ingredient and an effective amount of colloidal clay such as Attapulgite as a stabilizer to inhibit hydrolysis and/or isomerization and/or elimination and/or oxidation and/or re-crystallization, which comprises:
Forming a homogenous mixture by mixing the total quantity of said active ingredient with a portion of the total quantity of said colloidal clay such as Attapulgite, and at least one optionally excipient such as a binder, a diluent, a disintegrant and/or a glidant and mixing until uniform;—Sieving the above mixture through a sieve;
Adding to the sieved mixture the total quantities of at least one optionally excipient such as a binder, a diluent, a disintegrant and/or a glidant and mixing until uniform;
Admixing the remaining portion of the total quantity of said colloidal clay such as Attapulgite until uniform;—Subsequently, adding a lubricant and forming a homogenous mixture, and
Formulating the resulting mixture in a solid dosage form either by compressing it into a desired tablet form or by filling capsules or sachets.

15. A process for the preparation of a solid dosage form for oral administration, such as a tablet, a capsule or a sachet, containing a HMG-CoA reductase inhibitor or a pharmaceutical acceptable salt thereof as an active ingredient and an effective amount of colloidal clay such as Attapulgite as a stabilizer to inhibit hydrolysis and/or isomerization and/or elimination and/or oxidation and/or re-crystallization, which comprises:
forming a homogenous mixture by mixing the total quantity of said active ingredient with the total batch quantity of at least one optional excipient such as a binder, a diluent, a disintegrant, and/or a glidant and mixing until uniform; Sieving the above mixture through a sieve;
Adding to the sieved mixture the total quantity of said colloidal clay such as Attapulgite and mixing until uniform;—Subsequently, adding the total quantity of a lubricant and forming a homogenous mixture, and
Formulating the resulting mixture in a solid dosage form either by compressing it into a desired tablet form or by filling capsules o sachets.

16. The process according to claim 14, wherein said colloidal clay is Attapulgite.

17. The process according to claim 14, wherein said HMG-CoA reductase inhibitor is Atorvastatin or salt thereof.

18. The process according to claim 14, wherein said HMG-CoA reductase inhibitor is Fluvastatin or salt thereof.

19. The process according to claim 14, wherein said composition is immediate release.

20. The process according to claim 14, wherein said composition is sustained release.
